(19) United States
(12) Patent Application Publication
(10) Pub. No.: US 2002/0115996 A1
(43) Pub. Date: Aug. 22, 2002

(54) Endoscopic bi-polar clamping device

(75) Inventors: Jeffrey A. Wilson, Mendon, MA (US); John K. Danks, Delray Beach, FL (US); James R. Young, Ft. Landerdale, FL (US)

Correspondence Address: Donald N. Halgren, 35 Central Street, Manchester, MA, 01944, US

(73) Assignee: Pelican Medical Inc

(21) Appl. No.: 09/790,963

(22) Filed: Feb. 22, 2001

(51) Int. Cl. A61B 18/18

(52) U.S. Cl. 606/46; 606/50; 600/562

(57) ABSTRACT

The present invention relates to a clamping device for insertion through a working channel of an endoscope. The clamping device comprises an elongated housing body having a thumb ring on a first end thereof and a flexible elongated outer sheath attached to a second end of the housing. An inner sheath is slidably arranged within the housing and the outer sheath. A biasable clamp arrangement is connectively disposed on the distal end of the inner sheath. An annular ring is arranged on the distal end of the outer sheath. The biasable clamp arrangement is openable and closable in response to longitudinal movement of the inner sheath with respect to the outer sheath by a camming action between the arms and the annular ring. The arms may be connected to a proper circuit for providing mono-polar or bi-polar energization to tissue clamped between the arms comprising the clamp arrangement.

### BACKGROUND OF THE INVENTION

1. Field of the Invention

This invention relates generally to electro-coagulation of tissue in the body by a flexible instrument insertable through an endoscope.

2. Prior Art

Minimization of the time required to stop internal bleeding in a patient is critically important. An operating physician has several medical instruments in his/her armamentarium stopping such internal bleeding. In accordance with one modality that is particularly suited for bleeding in the gastrointestinal tract, a physician initially positions a flexible endoscope in the patient with its distal end proximate a hemorrhaging vessel. The physician may then insert an irrigator through a working channel in the endoscope to clear away the area by administering saline solution as a precursor to any attempts to stop the bleeding. The physician may then attempt to cauterize the bleeding vessel using a distally-positioned hemostat. Such instruments are constructed to be employed through a working channel of an endoscope to seal potential bleeding sites as in the gastrointestinal tract or the esophagus. Other hemostats use mono-electropolar electrodes, in which one electrode is carried by a catheter to a site or the other electrode is an exterior ground plate placed on a patient. A hemostat is an example of a device which supplies suitable current density and waveform of radio frequency energy to perform electro-coagulation or cauterization.

A hemostat of this nature may be utilized to burn out protrusions where the hemostat or probe is pushed against a polyp. However, it is often also pushed against healthy tissue, and ends up burning them both. When the hemostat is attempted to be removed, tissue may be pulled back and the charred tissue stays on it and the area then often re-bleeds.

It is an object of the present invention to overcome the disadvantages of the prior art.

It is a further object of the present invention to provide a clamping device which will minimize any lateral damage.

It is a further object of the present invention to provide a hemostatic device which utilizes a minimum of power to accomplish its goal.

It is yet a further object of the present invention, to provide a hemostatic device which is readily adaptable to multiple tissue configurations.

It is a further object of the present invention to provide a device with tip electrodes which permit the minimization of power requirements therefore.

It is still yet a further object of the present invention to provide a hemostatic device which is readily actuateable and clampable by the one-handed manipulation of the attending physician.

### BRIEF SUMMARY OF THE INVENTION

The present invention comprises a clamping device for use with, for example, a gastronomical endoscope. Such an endoscope typically has a working channel of about 2.8 millimeters. The clamping device of the present invention may be bi-polar, mono-polar, or of no polarity as discussed hereinbelow. The clamping device is an elongated structure having a first end with a thumb ring thereat connected to a first end of an elongated generally hollow housing. The housing has a distal, or second end, with a flexible outer sheath extending therefrom. The outer sheath is that portion of the clamping device which is fitted through a lumen in an endoscope during an endoscopic procedure.

A narrow inner sheath is slidedly disposed through the outer sheath. The inner sheath has a proximal end having an annular slider thereattached. The attachment between the slider and the inner sheath is accomplished through an elongated slot in the elongated housing. Sliding motion of the slider with respect to the elongated housing effects back and forth motion of the inner sheath with respect to the outer sheath.

In a first preferred embodiment of the present invention, at least one power cable is disposed through a lumen within the inner sheath, the power cable having a proximal end extending out a side of the slider and in communication through a switching arrangement, to a power source. The power source is arranged to provide variable power to the clamping device. The power cables extend to the distal end to the inner sheath and are in electrical communication with a female junction unit thereat.

A non-conducting ring of ceramic or plastic material is arranged within the distalmost end of the flexible outer sheath. The plug or ring at the distalmost end of the outer sheath has an obliquely angled inner surface defining its opened end.

A clamp arrangement is disposed through the opened end of the nonconductive plug. The clamp arrangement has a first or proximal end which is a male plug and a collar which mate into and about the female junction at the distalmost end of the inner sheath.

The clamp arrangement is comprised of a pair of articulable arms, each having a rounded or curved distalmost end. The arms are biasable toward and away from one another. Each arm has a mid-portion which is sloped and out of longitudinal alignment with their proximalmost shaft portions.

Rearward, or proximal motion of the slider towards the first end or thumb ring on the housing effects rearward withdrawal of the inner sheath with respect to the outer sheath. Rearward withdraw of the inner sheath effects a retraction of the clamps within the open distal end of the outer sheath. Movement of the clamps against the annular sloped cammed surface affects a camming action therebetween to affect the biasing of each respective arm towards one another as the clamps are retracted within the outer sheath.

In a further preferred embodiment, either clamp may be powered to effect a mono-polar capability to this clamping device. A further embodiment of the present invention comprises a power cable arranged through the flexible inner sheath through a separate lumen therewithin to provide bi-polar capabilities when the power source is energized and a switch is actuated, to deliver electrical current between the two arms when they are biased toward one another and particularly when they have a component of tissue or polyp therebetween.

The clamping arrangement is defined as aforesaid by a pair of arms with straight proximal portions secured within the collar and having male tips which are in electrical communication with the female receptacle with the distal end of the flexible inner sheath.

In a first preferred embodiment of the clamp arrangement, a first clamp is comprised of an elongated distalmost portion of stainless steel wire being reversed backwardly upon itself to define a post. The second clamp of the pair is paddle arrangement comprising an elongated loop, longer than the pad, the loop being a solid portion of conductive material. Reciprocally longitudinal motion of the clamp arrangement effects a biasing closed and open pivotal motion of the clamp arms with respect to one another.

A further embodiment of the clamp arrangement comprises the first clamp consisting of an elongated loop having an open space therebetween, and the second arm being comprised of an elongated loop with a conductive member or web spaced thereacross, defining a paddle. Another preferred embodiment of the present invention comprises the first arm having a paddle configuration, and the second arm also having a paddle configuration. A yet further preferred embodiment of the clamp arrangement of the present invention consists of the first arm having an elongated open loop in opposed juxtaposition with respect to the second arm which is also an elongated open loop thereadjacent. A yet further preferred embodiment of the clamp arrangement of the present invention consists of the first arm comprised of a pair of parallel fingers joined at a junction to the mid-portion of the clamp with their respective first and second opposed arms.

Thus, what has been shown is a clamping device capable of use through an endoscope, typically having a working channel of 2.8 millimeters, with a clamping arrangement on its distalmost end. The clamping arrangement being effective to isolate and cauterize polyps and tissue, from a variety of inter-changable clamping arrangements which clamps are miniaturized to concentrate rf energy to thereby permit that tissue treated to slough off without damaging adjacent tissue. The lateral damage to adjacent tissue is thus eliminated because the power utilized by the present invention due to the clamping arrangement may be kept to a minimum of about 20 watts, (⅓ to ¼ the typical power required of prior art devices) which is sufficient to coagulate tissue without charring and causing adherence of that tissue to the clamp. The power density is higher by virtue of the small clamp size, the clamp size being no greater than 2.8 millimeters in diameter. The exchangeable and interchangeable clamp tips with respect to the receiving plug at the distalmost end of the inner sheath effects rapid and advantageous use of a single device for various tissue configurations encountered. The clamping action affected by the camming action of the mid-portion of each respective clamp arm against the oblique cam surface of the nonconductive ring permits accurate control and response by the attending physician.

The invention thus comprises a clamping device for insertion through a working channel of an endoscope. The clamping device comprises an elongated housing body having a thumb ring on a first end thereof, a flexible elongated outer sheath attached to a second end of the housing, with an inner sheath slidably arranged within the housing and the outer sheath. A biasable clamp arrangement is connectively disposed on the distal end of the inner sheath, and an annular ring arranged on the distal end of the outer sheath, the biasable clamp arrangement being openable and closable in respect to longitudinal movement of the inner sheath with respect to the outer sheath. The clamp arrangement is comprised of a first and a second articulable arm. Each of the articulable arms have a respective mid-portion and a linear proximal portion, the mid-portion of each of the articulable arms being in skewed or non-parallel relationship with their respective linear portions thereof. At least one of the articulable arms is in electrical communication with a actuatable power source to provide a polarity to a body tissue portion pinched between the articulable arms when the arms are biased together. Both of the articulable arms may be in electrical communication with a actuatable power source to provide a bi-polar energy to a body tissue portion pinched between the articulable arms when the arms are biased together. The annular ring has an annular cam surface thereon, and the mid-portions of each of the arms have a sloped portion which engages the cam surface when the inner sheath is moved towards the proximal end of the housing. The clamping device has a proximal male end which is matable into a female junction at the distal end of the inner sheath. The first arm may comprise a post of wire and the second arm may be comprised of an elongated loop with a web of conductive material spaced thereacross to define a paddle, for clamping and electrically treating tissue therebetween. The post has a distalmost end and said paddle has a distalmost end, the post being distally shorter than the paddle. The first arm may be comprised of an elongated loop with an elongated open inner portion to define a loop arm, and the second arm comprises an elongated loop with a web of conductive material spaced thereacross to define a paddle, for clamping and electrically treating tissue therebetween. The first arm may be comprised of an elongated loop with a web of conductive material spaced thereacross to define a paddle, and wherein the second arm comprises an elongated loop with a web of conductive material spaced thereacross to define a paddle, for clamping tissue between flat surfaces and electrically treating that tissue therebetween. The first arm is comprised of an elongated loop with an elongated open central portion to define a loop arm, and wherein the second arm comprises an elongated loop with an open central portion to also define a loop arm, for clamping tissue between elongated open loop surfaces and electrically treating that tissue therebetween. The first arm is comprised of a pair of elongated parallel fingers with an elongated open space therebetween to define a first spaced-apart finger arm, and wherein the second arm also comprises a pair of elongated parallel fingers with an elongated open space therebetween to define a second spaced-apart finger arm, for clamping tissue between elongated spaced-apart linear fingers and electrically treating that tissue therebetween.

The invention also includes a method of treating mammalian tissue by a hollow, elongated endoscopic device having a distal end. The method may comprise the steps of: arranging a proximal end of a pair of removably replaceable clamps into a reciprocably movable female junction within the distal end of the endoscopic device; forming a sloped mid-portion of the clamps so as to be cammably biased by an electrically non-conductive ring arranged on the distal end of the elongated endoscopic device during reciprocable movement of the clamps with respect to the elongated endoscopic device; placing a mammalian tissue between the clamps; moving the female junction proximally with respect to the elongated endoscopic device to effect closure and engagement of the tissue between the clamps; and electrically energyzing at least one of the clamps to treat the tissue clamped therebetween. The method may include the steps of: removing at least one of the clamps from the female junction; and replacing the at least one of the clamps with a further clamp. The further clamp may have a distalmost tissue engaging end of different shaped configuration than the at least one of the clamps it replaced. The removably replaceable clamps may have dissimilar distalmost ends thereon with respect to one another, to permit movable interdigitation therebetween. The method may include the step of: shaping the distalmost ends of the clamps to permit a first of the clamps to enter the plane formed by the second of the clamps to permit tissue to be treated primarily in that plane of the second of those clamps.

### BRIEF DESCRIPTION OF THE DRAWINGS

The objects and advantages of the present invention will become more apparent when viewed in conjunction with the following drawings, in which:

FIG. 1 is a plan view of the clamping device and power circuit constructed according to the principles of the present invention;

FIG. 2 is a view taken along the lines 2-2 of FIG. 1;

FIG. 3 is a side-elevation view in partial section, showing a clamp arrangement in its distended or open configuration;

FIG. 4 is a side-elevational view similar to that shown in FIG. 3, with the clamp arrangement in a retracted and biased closed oriented configuration;

FIG. 5 is a perspective view of a further embodiment of the clamp arrangement showing a post and paddle configuration;

FIG. 6 is a perspective view of the clamp arrangement showing a loop and paddle clamp arrangement;

FIG. 7 is a perspective view of the clamp arrangement showing a pair of paddle arms;

FIG. 8 is a perspective view of a pair of arms comprised of an elongated loop; and

FIG. 9 is a perspective view of the clamp arrangement showing a pair of clamps with each arm comprised of a pair of parallel fingers.

### DETAILED DESCRIPTION OF THE PREFERRED EMBODIMENTS

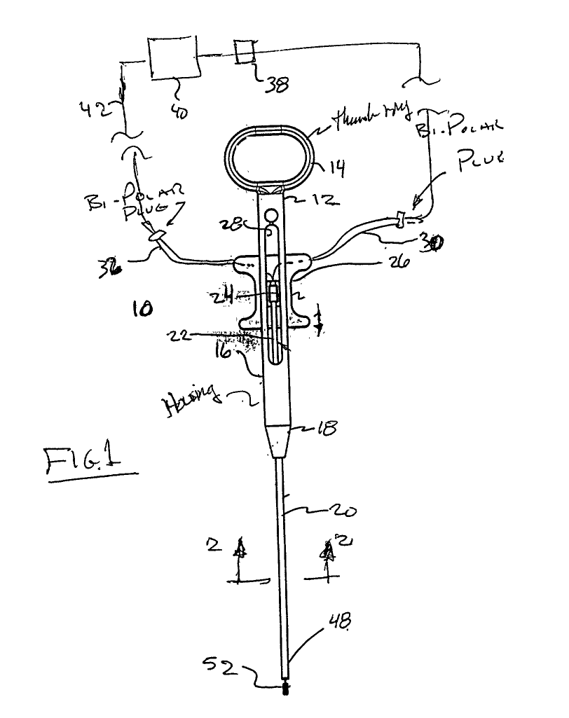

Referring now to the drawings in detail and particularly to FIG. 1, there is shown the present invention that comprises a clamping device 10 for use with, for example, a gastronomical endoscope. Such an endoscope typically has a working channel of about 2.8 millimeters. The clamping device 10 of the present invention may be bi-polar, mono-polar, or of no polarity as discussed hereinbelow. The clamping device 10 is an elongated structure having a thumb ring 14 thereat connected to a first end 12 of an elongated generally hollow housing 16. The housing 16 has a distal, or second end 18, with a flexible outer sheath 20 extending therefrom. The outer sheath 20 is that portion of the clamping device 10 which is fitted through a lumen in an endoscope during an endoscopic procedure.

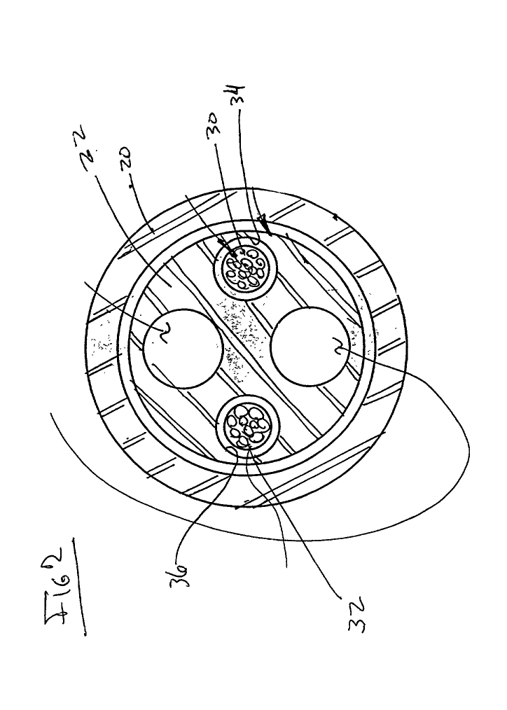

A narrow inner sheath 22 is slidedly disposed through the outer sheath 20, as shown in FIG. 2. The inner sheath 22 has a proximal end 24 having an annular slider 26 thereattached. The attachment between the slider 26 and the inner sheath 22 is accomplished through an elongated slot 28 in the elongated housing 16. Sliding motion of the slider 26 with respect to the elongated housing 16 effects back and forth motion of the inner sheath 22 with respect to the outer sheath 20.

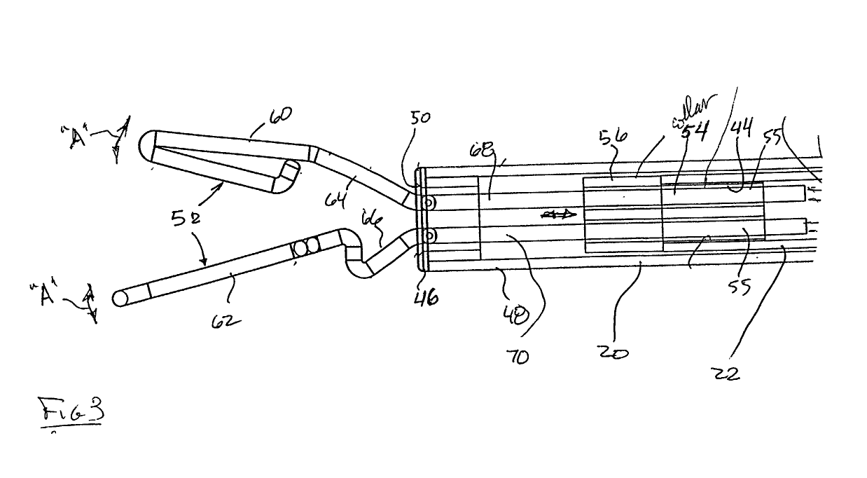

In a first preferred embodiment of the present invention, at least one power cable 30 and 32 is disposed through a lumen arrangement 34 and 36 within the inner sheath 22, as may be seen in FIG. 2. The power cable 30 and 32 each have a proximal end extending out a side of the slider 26 and in communication through a switching arrangement 38, to a power source 40 through a proper circuit 42. The power source 40 is arranged to provide variable power to the clamping device 10. The power cables 30 and 32 extend to the distal end to the inner sheath 22 and are in electrical communication with a female junction unit 44 thereat, as may be seen in FIG. 3.

A non-conducting ring 46, made of ceramic or plastic material is arranged within the distalmost end 48 of the flexible outer sheath 20. The plug or ring 46 at the distalmost end 48 of the outer sheath 20 has an obliquely angled inner surface 50 defining its opened end.

A clamp arrangement 52 is disposed through the opened end of the non-conductive ring 46. The clamp arrangement 52 has a first or proximal end 54 which is a male plug and a has collar 56 which mate into and about the female junction 44 at the distalmost end of the inner sheath 22.

The clamp arrangement 52 is comprised of a pair of articulable arms 60 and 62, each having a rounded or curved distalmost end “D”. The arms 60 and 62 are biasable toward and away from one another as indicated by the arrows “A”, shown in FIG. 3. Each arm 60 and 62 has a mid-portion 64 and 66 each of which is respectively sloped and out of longitudinal alignment with their respective proximalmost shaft portions 68 and 70.

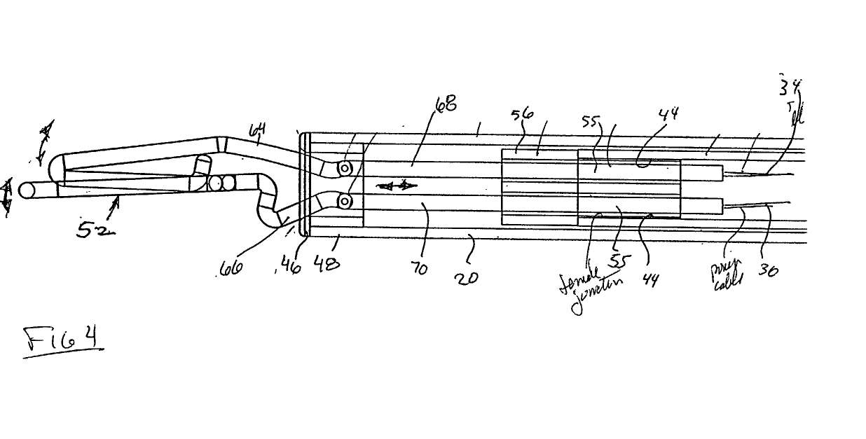

Rearward, or proximal motion of the slider 26 towards the first end or thumb ring 14 on the housing 16, as indicated by the arrow “R” in FIG. 1 effects rearward withdrawal of the inner sheath 22 with respect to the outer sheath 20. Rearward withdraw of the inner sheath 22 effects a retraction of the clamp arrangement 52 within the open distal end of the outer sheath 20, as evidenced between the clamp arrangement 52 position between FIG. 3 and FIG. 4. Movement of the mid-portions of the clamps 60 and 62 against the annular sloped cammed surface 50 effects a camming action therebetween to affect the biasing of each respective arm 60 and 62 towards one another as the clamp arrangement 52 is retracted within the outer sheath 20.

In a further preferred embodiment, either clamp 60 or 62 may be powered to effect a mono-polar capability to this clamping device 10. A further embodiment of the present invention comprises the pair of power cables 30 and 32 arranged through the flexible inner sheath 22 through a separate lumens 34 and 36 therewithin to provide the bi-polar capabilities when the power source 40 is energized and the switch 38 is actuated, to deliver electrical current between the two arms 60 and 62 when they are biased toward one another and particularly when they have a component of tissue or polyp therebetween.

The clamping arrangement 52 is thus defined as aforesaid by a pair of arms 60 and 62 with straight proximal portions 68 and 70 secured within the collar 54 and having male tips 55 which are in electrical communication with the female receptacle 44 with the distal end of the flexible inner sheath 22, as shown in FIGS. 3 and 4.

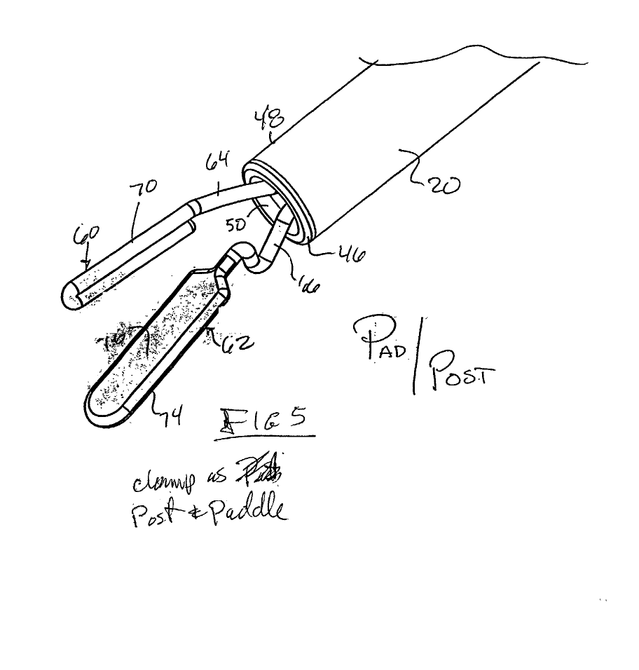

In a first preferred embodiment of the clamp arrangement 52, a first clamp 60 is comprised of an elongated distalmost portion of stainless steel wire being reversed backwardly upon itself to define a post 70, as shown in FIG. 5. The second clamp 62 of the clamp arrangement pair 52 is paddle arrangement 72 comprising an elongated loop 74, longer than the post 70, the loop 74 having a solid central portion 76 of conductive material, as also shown in FIG. 5. Reciprocal longitudinal motion of the clamp arrangement 52 effects a biasing closed and open pivotal motion of the clamp arms with respect to one another, as shown between the orientation of the first and second clamps 60 and 62, in FIGS. 3 and 4.

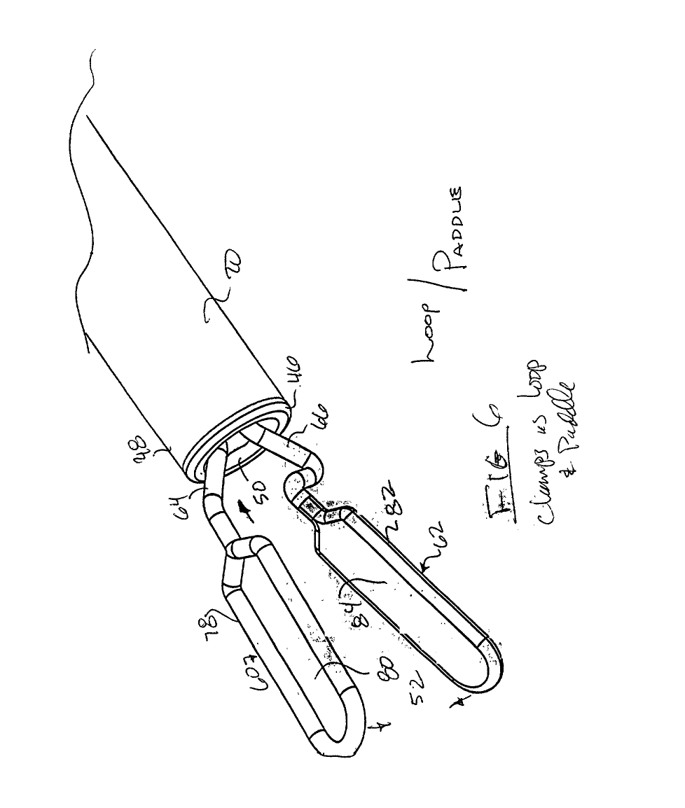

A further embodiment of the clamp arrangement 52, as shown in FIG. 6, comprises the first clamp 60 consisting of an elongated loop 78 having an open space 80 therebetween. The second arm 62 is comprised of an elongated loop 82 with a conductive member or web 84 spaced thereacross, defining a paddle.

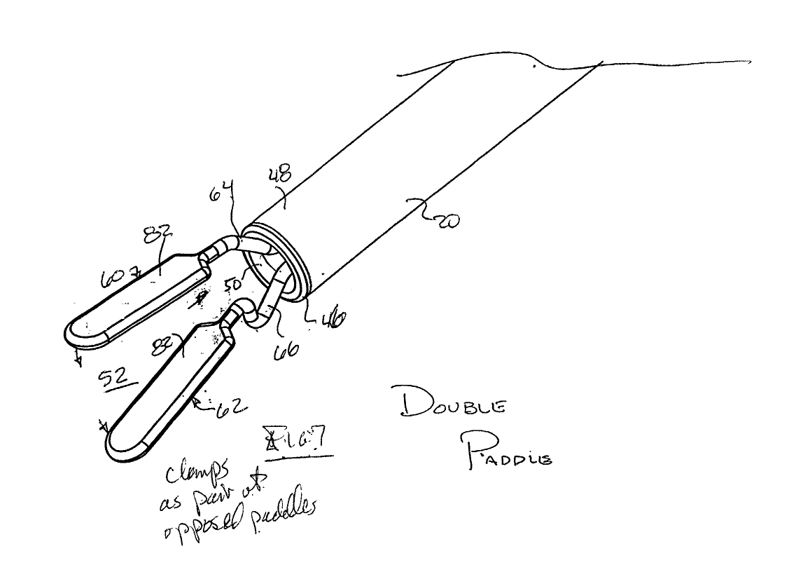

Another preferred embodiment of the present invention of the clamp arrangement 52 is shown in FIG. 7, which comprises the first arm 60 having a paddle configuration 82, and the second arm 62 also having a paddle configuration 82, as was described for only the second arm 62 shown in the embodiment disclosed in FIG. 6.

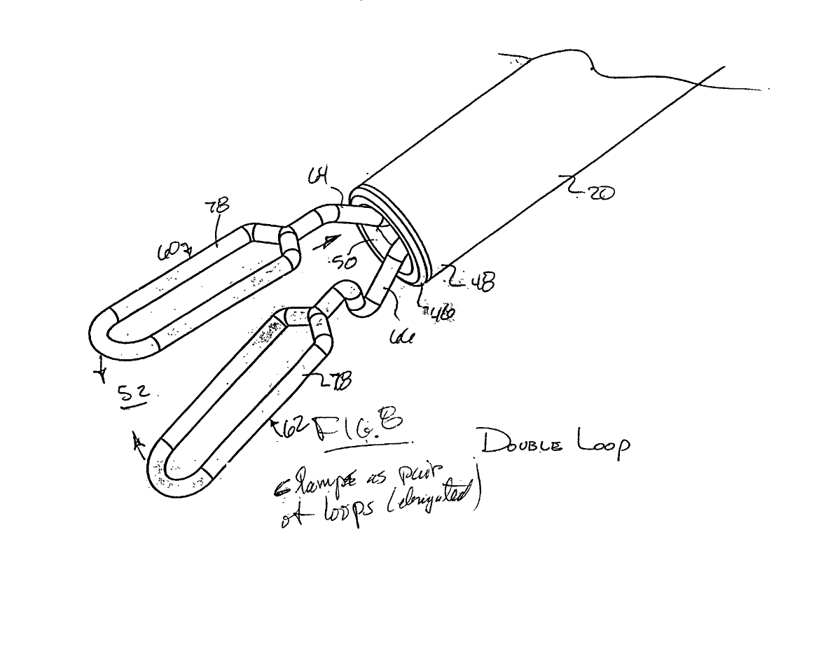

A yet further preferred embodiment of the clamp arrangement 52 of the present invention consists of the first arm 60 having an elongated open loop 78 in opposed juxtaposition with respect to the second arm 62 which is also an elongated open loop 78 thereadjacent, as shown in FIG. 8, which loop 78 was shown and described for only the first arm 62 in FIG. 6.

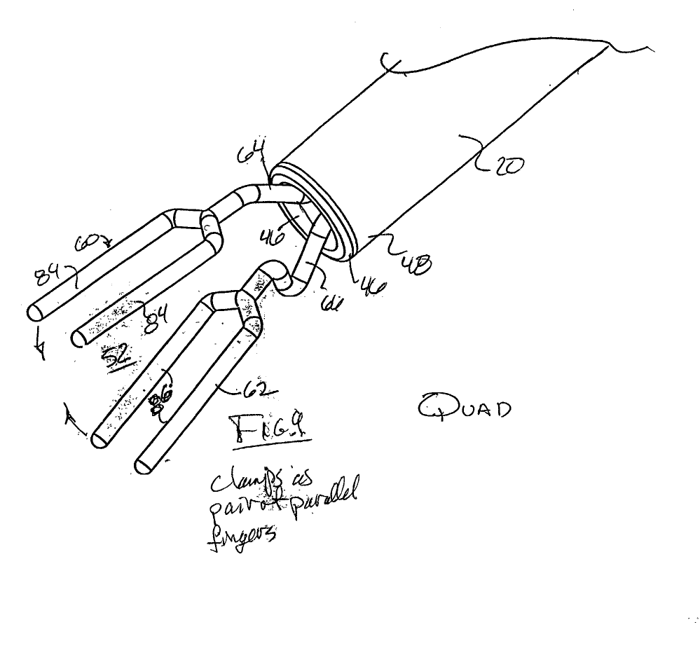

A yet farther preferred embodiment of the clamp arrangement 52 of the present invention is shown in FIG. 9, which consists of the first arm 60 comprised of a pair of parallel fingers 84 joined at a junction 86 to the mid-portion 64 of the clamp arrangement 52, and the second arm 62 similarly configured with parallel fingers 86, closable in parallel juxtaposition with respect to the fingers 84 of the first arm 60 to permit tissue to be clamped therebetween those respective first and second opposed arms 60 and 62.

Thus, what has been shown is a clamping device capable of use through an endoscope, typically having a working channel of 2.8 millimeters, with a clamping arrangement on its distalmost end. The clamping arrangement being effective to isolate and cauterize polyps and tissue, from a variety of inter-changable clamping arrangements which clamps are miniaturized to concentrate rf energy to thereby permit that tissue treated to slough off without damaging adjacent tissue. The lateral damage to adjacent tissue is thus eliminated because the power utilized by the present invention due to the clamping arrangement may be kept to a minimum of about 20 watts, (⅓ to ¼ the typical power required of prior art devices) which is sufficient to coagulate tissue without charring and causing adherence of that tissue to the clamp. The power density is higher by virtue of the small clamp size, the clamp size being no greater than 2.8 millimeters in diameter. The exchangeable and interchangeable clamp tips with respect to the receiving plug at the distalmost end of the inner sheath effects rapid and advantageous use of a single device for various tissue configurations encountered. The clamping action affected by the camming action of the mid-portion of each respective clamp arm against the oblique cam surface of the nonconductive ring permits accurate control and response by the attending physician.

We claim:

1. A clamping device for insertion through a working channel of an endoscope, said clamping device comprising:
an elongated housing body having a thumb ring on a first end thereof;
a flexible elongated outer sheath attached to a second end of said housing, with an inner sheath slidably arranged within said housing and said outer sheath;
a biasable clamp arrangement connectively disposed on the distal end of said inner sheath, and an annular ring arranged on the distal end of said outer sheath, said biasable clamp arrangement being openable and closable in respect to longitudinal movement of said inner sheath with respect to said outer sheath.

2. The clamping device as recited in claim 1, wherein said clamp arrangement is comprised of a first and a second articulable arm, each of said articulable arms having a respective mid-portion and a linear proximal portion, said mid-portion of each of said articulable arms being in skewed or non-parallel relationship with their respective linear portions thereof to facilitate relative motion therebetween.

3. The clamping device as recited in claim 2, wherein at least one of said articulable arms is in electrical communication with a actuatable power source to provide a polarity to a body tissue portion pinched between said articulable arms when said arms are biased together.

4. The clamping device as recited in claim 2, wherein both of said articulable arms are in electrical communication with a actuatable power source to provide a bi-polar energy to a body tissue portion pinched between said articulable arms when said arms are biased together.

5. The clamping device as recited in claim 2, wherein said annular ring has an annular cam surface thereon, and said mid-portions of each of said arms have a sloped proximalmost portion which engages said cam surface when said inner sheath is moved towards the proximal end of said housing.

6. The clamping device as recited in claim 2, wherein said clamping device has a proximal male end which is matable into a female junction at said distal end of said inner sheath.

7. The clamping device as recited in claim 2, wherein said first arm comprises a post of wire and said second arm is comprised of an elongated loop with a web of conductive material spaced thereacross to define a paddle, for clamping and electrically treating tissue engaged therebetween.

8. The clamping device as recited in claim 7, wherein said post has a distalmost end and said paddle has a distalmost end, said post being distally shorter than said paddle.

9. The clamping device as recited in claim 2, wherein said first arm is comprised of an elongated loop with an elongated open inner portion to define a loop arm, and said second arm comprises an elongated loop with a web of conductive material spaced thereacross to define a paddle, for clamping and electrically treating tissue engaged therebetween.

10. The clamping device as recited in claim 2, wherein said first arm is comprised of an elongated loop with a web of conductive material spaced thereacross to define a paddle, and wherein said second arm comprises an elongated loop with a web of conductive material spaced thereacross to define a paddle, for clamping tissue between flat surfaces and electrically treating that tissue therebetween.

11. The clamping device as recited in claim 2, wherein said first arm is comprised of an elongated loop with an elongated open central portion to define a loop arm, and wherein said second arm comprises an elongated loop with an open central portion to also define a loop arm, for clamping tissue between elongated open loop surfaces and electrically treating that tissue therebetween.

12. The clamping device as recited in claim 2, wherein said first arm is comprised of a pair of elongated parallel fingers with an elongated open space therebetween to define a first spaced-apart finger arm, and wherein said second arm also comprises a pair of elongated parallel fingers with an elongated open space therebetween to define a second spaced-apart finger arm, for clamping tissue between elongated spaced-apart linear fingers and electrically treating that tissue therebetween.

13. A method of treating mammalian tissue by a hollow, elongated endoscopic device having a distal end, said method comprising the steps of:
arranging a proximal end of a pair of removably replaceable clamps into a reciprocably movable female junction within said distal end of said endoscopic device;
forming a sloped mid-portion of said clamps so as to be cammably biased by an electrically non-conductive ring arranged on said distal end of said elongated endoscopic device during reciprocable movement of said clamps with respect to said elongated endoscopic device;
placing a mammalian tissue between said clamps;
moving said female junction proximally with respect to said elongated endoscopic device to effect closure and engagement of said tissue between said clamps; and
electrically energyzing at least one of said clamps to treat said tissue clamped therebetween.

14. The method as recited in claim 13, including the step of:
removing at least one of said clamps from said female junction;
and
replacing said at least one of said clamps with a further clamp.

15. The method as recited in claim 14, wherein said further clamp has a distalmost tissue engaging end of different shaped configuration than said at least one of said clamps it replaced.

16. The method as recited in claim 14, wherein said removably replaceable clamps have dissimilar distalmost ends thereon with respect to one another, to permit movable interdigitation therebetween.

17. The method as recited in claim 16, including the step of:
shaping said distalmost ends of said clamps to permit a first of said clamps to enter the plane formed by the second of said clamps to permit tissue to be treated primarily in that plane of the second of those clamps.
